(19) United States
(12) Patent Application Publication
(10) Pub. No.: US 2014/0371698 A1
(43) Pub. Date: Dec. 18, 2014

(54) SOUND ABSORBING NON-WOVEN MATERIAL, SOUND ABSORBING MULTILAYER FILM, AND LAMINATES MADE THEREOF

(71) Applicant: Hollister Incorporated, Libertyville, IL (US)

(72) Inventors: Moh-Ching Oliver Chang, Lake in the Hills, IL (US); Kimberly R. Hansford, Winthrop Harbor, IL (US)

(21) Appl. No.: 14/368,220

(22) PCT Filed: Dec. 28, 2012
(86) PCT No.: PCT/US12/71953

§ 371 Date: Jun. 23, 2014

Related U.S. Application Data
(60) Provisional application No. 61/580,885, filed on Dec. 28, 2011.

(51) Int. Cl. A61F 5/44; A61L 28/00; A61F 5/441

(52) U.S. Cl. CPC A61F 5/4404; A61F 5/441; A61L 28/0011
USPC 604/333; 604/332; 252/62; 525/313; 181/294

(57) ABSTRACT

An ostomy appliance includes a sound absorbing film comprising a triblock copolymer including polystyrene blocks and vinyl-bond rich rubber mid-block, and a sound absorbing non-woven material comprising the triblock copolymer including polystyrene blocks and vinyl-bond rich rubber mid-block. The ostomy appliance has improved sound absorbing properties to insulate flatulence noise.

### BACKGROUND

The present disclosure relates to a sound absorbing material, and more particularly to non-woven materials and multilayer films having sound absorbing properties for medical uses such as ostomy applications.

Ostomy appliances for collecting body waste, such as ostomy pouches, are used by patients who have had surgery such as a colostomy, ileostomy, or urostomy. When body waste is released from a stoma, flatus gas is often released together with the waste. The flatus gas passing the stoma can cause a vibratory transient in body tissue, which is uncontrollable by the patient. Such release of the flatus gas from the stoma can accompany indiscreet noise, which can cause embarrassment to the patient.

Ostomy pouches comprising a quiet film to reduce noise produced by ostomy pouches, for example, plastic crackling sound made by the ostomy pouch when a user moves around, have been developed. Examples of such a quiet film include multilayer films disclosed in U.S. Pat. No. 7,270,860, which is assigned to the assignee of the present application and incorporated herein in its entirety by reference. However, these quiet films are insufficient to insulate the flatus noise to prevent embarrassment.

Because of the inherent severe medical, social, and personal concerns related to the need for use of an ostomy appliance, improvements in ostomy appliances are desired. Any appreciable improvement in such ostomy appliances to provide greater discretion and privacy is of great importance in the quality of life of the increasing number of ostomy patients. The present disclosure provides improved ostomy appliances according to various embodiments to enhance sound insulating properties of such ostomy appliances.

### BRIEF SUMMARY

Sound absorbing non-woven materials, sound absorbing multilayer films, and laminates thereof, which are configured to insulate flatulence gas noise in ostomy appliances, are provided according to various embodiments of the present disclosure. The sound absorbing non-woven material comprises fibers including a triblock copolymer including polystyrene blocks and vinyl-bond rich rubber mid-block. The sound absorbing multilayer film comprises at least one layer including a triblock copolymer including polystyrene blocks and vinyl-bond rich rubber mid-block. The sound absorbing non-woven material and the sound absorbing multilayer film may be directly laminated by heat sealing or laminated together using an adhesive or any other known laminating methods, and such a sound absorbing laminate is used to make an ostomy pouch to provide excellent sound insulation properties. The sound absorbing non-woven material and the sound absorbing multilayer film can also be used on their own for ostomy appliances.

In one aspect, a sound insulating ostomy pouch is provided. The sound insulating ostomy pouch includes a first wall, a second wall, and at least one sound absorbing non-woven layer. The first wall and the second wall are sealed along their peripheral edges to define a cavity. The at least one sound absorbing non-woven layer is heat sealed to an adjacent surface of the first wall or the second wall about its entire surface area to form a thermal laminate. The at least one sound absorbing non-woven layer may include fibers containing a non-hydrogenated vinyl-bond rich styrene-isoprene-styrene (SIS) block copolymer.

In some embodiments, at least one of the first wall and the second wall is formed from a sound absorbing film including the non-hydrogenated vinyl-bond rich SIS block copolymer. Further, the sound absorbing film can be a multilayer film including at least one layer containing more than 50% wt. of the non-hydrogenated vinyl-bond rich SIS block copolymer. In one embodiment, the sound absorbing film is a seven-layer film including an odor barrier layer, two tie layers, two inner layers, and two skin layers, in which each of the skin layers contains more than 50% wt. of the non-hydrogenated vinyl-bond rich SIS block copolymer. Further, each of the inner layers can contain a triblock copolymer. For example, each of the inner layers contains more than 50% wt. of the non-hydrogenated vinyl-bond rich SIS block copolymer. Alternatively, the sound absorbing film can be a multilayer film including at least one layer containing a hydrogenated vinyl-bond rich SIS block copolymer.

In some embodiments, the fibers are formed from a blend of the non-hydrogenated vinyl-bond rich SIS block copolymer and a polyethylene or a polypropylene. In other embodiments, the fibers have a sheath/core construction, of which the sheath comprises a polyethylene or a polypropylene, and the core comprises the non-hydrogenated vinyl-bond rich SIS block copolymer.

In one embodiment, the pouch includes a first sound absorbing non-woven layer and a second sound absorbing non-woven layer, in which a surface of the first sound absorbing non-woven layer is heat sealed to an adjacent surface of the first wall along their entire surface areas, and a surface of the second sound absorbing non-woven layer is heat sealed to an adjacent surface of the second wall along their entire surface areas (each wall comprises a thermal laminate including a layer of sound absorbing non-woven material). Each of the first wall and the second wall contains the non-hydrogenated vinyl-bond rich SIS block copolymer.

In another aspect, a sound insulating ostomy pouch including a first wall, a second wall, and at least one sound absorbing non-woven layer which is attached to the first wall is provided. The first wall and the second wall are sealed along their peripheral edges to define a cavity. The at least one sound absorbing non-woven layer includes fibers formed from a polymer blend comprising a vinyl-bond rich triblock copolymer.

In some embodiments, at least one of the first wall and the second wall is formed from a sound absorbing film including a vinyl-bond rich triblock copolymer. Further, the sound absorbing film can be a multilayer film including at least one layer containing more than 50% wt. of a non-hydrogenated vinyl-bond rich SIS block copolymer. In one embodiment, the sound absorbing film is a seven-layer film including an odor barrier layer, two tie layers, two inner layers, and two skin layers, in which each of the skin layers contains more than 50% wt. of the non-hydrogenated vinyl-bond rich SIS block copolymer. Further, each of the inner layers can contain a vinyl-bond rich triblock copolymer. For example, each of the inner layers contains more than 50% wt. of the non-hydrogenated vinyl-bond rich SIS block copolymer.

In some embodiments, the fibers are formed from a polymer blend comprising a non-hydrogenated vinyl-bond rich SIS block copolymer and a polyethylene or a polypropylene. In other embodiments, the fibers are formed from a polymer blend comprising about 20 wt. % to about 70 wt. % of a hydrogenated vinyl-bond rich SIS block copolymer. For example, the fibers are formed from a polymer blend comprising about 30 wt. % of the hydrogenated vinyl-bond rich SIS block copolymer and about 70 wt. % of a styrene-ethylene/propylene-styrene (SEPS) block copolymer. In another example, the fibers are formed from a polymer blend comprising a hydrogenated vinyl-bond rich SIS block copolymer and at least one polyolefin, such as polyethylene or polypropylene.

In one embodiment, the pouch includes a first sound absorbing non-woven layer and a second sound absorbing non-woven layer, in which a surface of the first sound absorbing non-woven layer is heat sealed to an adjacent surface of the first wall along their entire surface areas, and a surface of the second sound absorbing non-woven layer is heat sealed to an adjacent surface of the second wall along their entire surface areas (each wall comprises a thermal laminate including a layer of sound absorbing non-woven material). Each of the first sound absorbing non-woven layer and the second sound absorbing non-woven layer includes fibers formed from a polymer blend comprising a hydrogenated vinyl-bond rich SIS block copolymer. In another embodiment, the at least one sound absorbing non-woven layer is attached to the pouch wall via an adhesive.

The ostomy pouch in accordance with any of above discussed embodiments is configured to insulate flatulence noise, in which each of the first wall and the second wall is configured to absorb the flatulence noise, and each of the at least one sound absorbing non-woven layers is configured to absorb the flatulence noise.

In another aspect, a sound absorbing non-woven material for ostomy appliances is provided. The sound absorbing non-woven material includes fibers comprising a non-hydrogenated vinyl-bond rich SIS block copolymer.

In some embodiments, the sound absorbing non-woven material comprises fibers formed from a blend of the non-hydrogenated vinyl-bond rich SIS block copolymer, and a polyethylene or a polypropylene. In other embodiments, the sound absorbing non-woven material comprises fibers having a sheath/core construction, in which the sheath is formed of a polyethylene or a polypropylene, and the core is formed of the non-hydrogenated vinyl-bond rich SIS block copolymer. The non-woven material can have a basis weight of between about 20 grams per square meter (gsm) and about 60 gsm of fibers. In one embodiment, the non-woven material has a basis weight of between about 30 gsm and 50 gsm.

In yet another aspect, a sound absorbing non-woven material for ostomy appliances including fibers formed from a polymer blend comprising a hydrogenated vinyl-bond rich SIS block copolymer is provided. In some embodiments, the fibers are formed form a polymer blend comprising about 20 wt. % to about 70 wt. % of the hydrogenated vinyl-bond rich SIS block copolymer. For example, the fibers are formed from a polymer blend comprising about 30 wt. % of the hydrogenated vinyl-bond rich SIS block copolymer and about 70 wt. % of a SEPS block copolymer. Preferably, the sound absorbing non-woven material has a basis weight between about 20 gsm and about 60 gsm of fibers.

In one aspect, a sound absorbing film for ostomy appliances is provided. The sound absorbing film includes at least one layer comprising at least 50% wt. of a non-hydrogenated vinyl-bond rich SIS block copolymer. In some embodiments, the sound absorbing film is a multilayer film including an odor barrier layer and a first skin layer and a second skin layer. In such embodiments, at least one of the first skin layer and the second skin layer comprises at least 50% wt. of the non-hydrogenated vinyl-bond rich SIS block copolymer. Further, the multilayer film can also include a first inner layer and a second inner layer, in which at least one of the inner layers includes a vinyl-bond rich triblock copolymer. In one embodiment, at least one of the first inner layer and the second inner layer comprises at least 50% wt. of the non-hydrogenated vinyl-bond rich SIS block copolymer. In another embodiment, the sound absorbing film is a seven-layer film including an odor barrier layer, two tie layers, two inner layers, and two skin layers, of which each of the skin layers contains at least 50% wt. of the non-hydrogenated vinyl-bond rich SIS block copolymer, and each of the inner layers contains at least 50% wt. of the non-hydrogenated vinyl-bond rich SIS block copolymer.

In another aspect, a sound absorbing multilayer film for ostomy appliance is provided. The sound absorbing multilayer film includes at least one layer comprising a hydrogenated vinyl-bond rich SIS block copolymer. In some embodiments, the sound absorbing multilayer film includes an odor barrier layer and a first skin layer and a second skin layer, in which at least one of the skin layers comprises the hydrogenated vinyl-bond rich SIS block copolymer. The multilayer film can also include a first inner layer and a second inner layer, in which at least one of the inner layers comprises the hydrogenated vinyl-bond rich SIS block copolymer.

Other aspects, objectives and advantages will become more apparent from the following detailed description when taken in conjunction with the accompanying drawings.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a cross-sectional illustration of an ostomy appliance including a pouch and a skin barrier in accordance with an embodiment of the present disclosure;

FIG. 2 is a cross-sectional illustration of a sound absorbing multilayer film according to an embodiment;

FIG. 3 is a perspective illustration of a fiber having a sheath/core construction for a sound absorbing non-woven material according to an embodiment; and

FIG. 4 is a graph showing sound transmission loss data for laminate samples.

### DETAILED DESCRIPTION

While the present disclosure is susceptible of embodiment in various forms, there is shown in the drawings and will hereinafter be described a presently preferred embodiment with the understanding that the present disclosure is to be considered an exemplification and is not intended to limit the disclosure to the specific embodiment illustrated. The words “a” or “an” are to be taken to include both the singular and the plural. Conversely, any reference to plural items shall, where appropriate, include the singular.

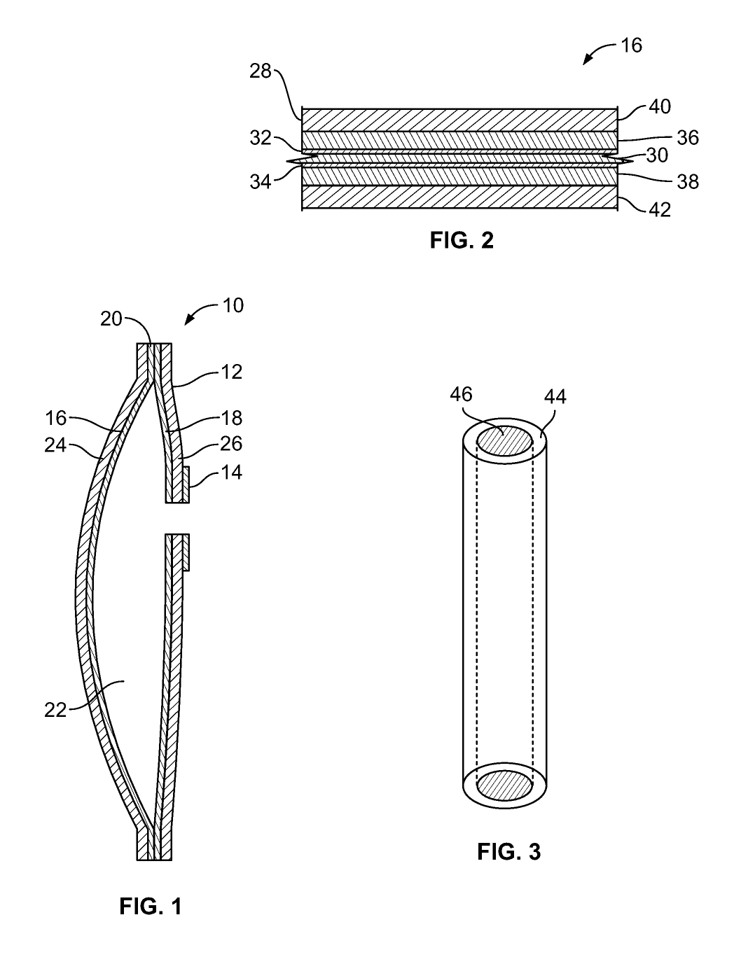

FIG. 1 is a cross-sectional illustration of a one-piece ostomy appliance 10 according to an embodiment. The ostomy appliance generally includes a pouch 12 and a skin barrier 14. The pouch 12 includes first and second opposing walls 16, 18, which are sealed around peripheral edges 20 thereof by heat sealing or by any other suitable means to define a cavity 22 therebetween. The pouch 12 also includes a first non-woven layer 24 attached to the first wall 16, and a second non-woven layer 26 attached to the second wall 18.

The first and second walls 16, 18 are formed from a suitable polymeric film, and each of the walls can be formed from a same film or different films. The films can be a single layer film or a multilayer film. Preferably, the first and second walls 16, 18 are formed from a sound absorbing multilayer film including at least one layer having sound absorbing properties. In one embodiment, the first and the second walls 16, 18 are formed of a seven-layer film.

FIG. 2 shows a cross sectional view of the first wall 16 formed from the seven-layer film 28. Although not shown, the second wall 18 is also formed of the same seven-layer film 28 in this embodiment. The seven-layer film 28 includes a barrier 30, tie layers 32, 34, inner layers 36, 38, and skin layers 40, 42. The barrier layer 30 can be formed of a suitable film having gas barrier properties. Preferably, the barrier layer 30 is formed from a non-chlorine containing polymer that is substantially impermeable to malodor causing compound typically encountered in ostomy pouches. Such malodor causing compounds can include sulfur containing compounds and indoles.

On either side of the barrier layer 30 is the tie layer 32, 34. The tie layers facilitate adhesion of the barrier layer to the remainder of the film structure. The first and second inner layers 36, 38 are adjacent to the tie layers 32, 34, respectively. Preferably, the inner layers impart tear strength to the film, while at the same time facilitate achieving sound absorbing properties of the film. The skin layers 40, 42, that are adjacent the first and second inner layers 36, 38, respectively. The skin layers 40, 42 provide good heat sealing characteristics to form a pouch and to be heat laminated with the non-woven layers 24, 26. Preferably, the skin layers 40, 42 also provide sound absorbing properties of the film. The seven-layer film 28 thus has the structure ABCDCBA, where A represents the skin layers, B represents the inner layers, C represents the tie layers, and D represents the barrier layer. Although the film 28 of this embodiment includes seven-layers, in other embodiments, a multilayer barrier film can include more than seven-layers or less than seven-layers. For example, a multilayer film according to this disclosure can be a six-layer film including a barrier layer, two tie layers, an inner layer, and two skin layers (i.e. ABCDCA), or alternatively, a five-layer film including a barrier layer, two tie layers and two outer layers (i.e. ACDCA, BCDCB or ACDCB).

The barrier layer 30 can be formed from various materials. Suitable barrier layer materials include resins such as amorphous polyamide (nylon) resin, and an anhydride-modified olefinic polymer or copolymer, or an epoxy modified olefin polymer or copolymer. In this embodiment, the barrier layer 30 is formed from a blend of an amorphous polyamide, such as Selar® PA3426R, by DuPont Company, and a functionalized rubber blend or compound, such as Lotader® 4720.

The tie layers 32, 34 can be formed of the same material or different materials. In the embodiment of FIG. 2, each of the tie layers 14, 16 is formed from a maleated ethylene methyl acrylate copolymers (EMA) having maleic anhydride present at about 0.3 percent and methyl acrylate present at about 20 percent of the resin. One such material is available from Arkema, Inc. as Lotader®4503.

The first and second inner layers, 36, 38 can be formulated from the same material or different materials. In the embodiment of FIG. 2, each of the first and second inner layers 36, 38 is formed of the same material. The inner layers 36, 38 impart mechanical (tear) strength to the film 28. Ethylene based polymers, such as ethylene vinyl acetate (EVA) copolymer, ethylene-octene (EO) plastomers, and ethylene-propylene (EP) copolymers (PP-elastomer) are suitable film forming materials for the inner layers. One suitable material is an ethylene vinyl acetate (EVA) copolymer having a vinyl acetate content of about 8 percent to 30 percent and preferably about 10 percent to about 25 percent, a melting point temperature of about 86° C. and a Shore A hardness of about 91, such as Escorene®FL00218, available from ExxonMobil Corporation.

Another suitable material is an EO plastomer having a melting point temperature of about 95° C. and specific gravity of about 0.902, such as Exact® 0203 resin, also available from ExxonMobil Corporation, which has a specific gravity of about 0.88 and a Shore A hardness of about 95. This resin is designed for both monolayer and multilayer co-extruded cast film applications and is suitable in applications that require toughness and heat sealing performance. Typical applications include film for industrial packaging.

Still another suitable resin is an ethylene-propylene copolymer (PP-elastomer) resin that exhibits a low melt flow rate making it suitable for film application and heat sealing. It has a low modulus and thus exhibits low noise characteristics. It has excellent compatibility with polypropylene (PP) and polyethylene (PE). One such material is available from Dow Chemical from as Versify®2200. This resin has melting point of about 82° C., a Shore A hardness of 94 and a Shore D hardness of 42. It has a specific gravity of 0.878. Blends of various PP copolymer resins have also been found to be suitable, for example, blends of Versify®2200 and Versify®3400, which is a similar PP copolymer resin, but has a higher melting point of about 97° C., a Shore A hardness of 72 and a Shore D hardness of 22, and a specific gravity of about 0.865. Suitable blends can have ratios of about 50 percent of Versify® 2200 to about 75 percent of Versify® 2200 by weight of the blend. PP-elastomers such as Versify® from Dow, Vistamaxx® from Exxon, and Notio® from Mitsui are also suitable.

In a preferred embodiment, the inner layers 36, 38 also provide at least some sound absorbing properties. In such an embodiment, each of the inner layers 36, 38 comprises a vinyl-bond rich triblock copolymer, such as Hybrar® from Kuraray Co. Ltd., to enhance mechanical properties and sound absorbing properties of the film. For example, the each of inner layers 36, 38 is formed from a blend of a hydrogenated vinyl-bond rich styrene-isoprene-styrene block copolymer (SEPS, e.g. Hybrar® 7125) and a PP-elastomer)(Vistamaxx®).

The skin layers 40, 42 can likewise be formed of the same or different materials. In the embodiment of FIG. 2, the skin layers 40, 42 are formed of the same material. The skin layers 40, 42 are formed of a material having suitable heat sealability, such that the skin layers can be heat sealed to form a pouch and/or can be heat sealed to non-woven materials. Suitable materials for the skin layers 40, 42 include, for example, copolymers of ethylene with vinyl esters, such as vinyl acetate copolymer (EVA) and copolymers of ethylene methyl acrylate (EMA). EVA copolymers contain about 10 percent to 35 percent vinyl acetate and more preferably, about 18 percent by weight vinyl acetate, by weight of the copolymer. One material is available from ExxonMobil as product Escorene® Ultra FL00218. Such a material has a melting point temperature of 86° C. and a Shore A hardness of about 91. EVA based materials provide increased comfort for the person using an ostomy pouch made from this material. EVA is also known to exhibit the necessary characteristics for joining to another EVA member, as by heat sealing, to provide an air-tight, liquid-tight seal at the joint or seal. EVA materials can be blended to facilitate formation and film extrusion. For example, an EVA blend can have about 98 percent by weight EVA with about 2 percent anti-block and slip additives, in an EVA carrier. One suitable additive is available from A. Schulman Inc., as Polybatch® SAB-1982VA.

EMA copolymers include about 10 to about 35 percent of the methyl acrylate and preferably about 18.5 percent to about 30 percent by weight methyl acrylate, by weight of the copolymer. Such EMA copolymers typically have melting point temperatures of about 85° C. to 87° C. and a Shore A hardness of about 73 and Shore D hardness of about 20 to 25. Suitable materials are available from Arkema Inc. as Lotryl® 18AM02 and from DuPont as Elvaloy® 1330AC. The EMA resins can also be blended with anti-block and slip additives in an EVA carrier. One suitable material for blending is the aforementioned Polybatch® SAB-1982VA. Such a blend can have, for example EMA at about 98 percent by weight, with about 2 percent Polybatch® SAB-1982VA anti-block and slip additive.

As set forth above, other suitable seal and skin layers are formed as a blend of EVA copolymer (Escorene® FL00218 present at 49 percent) and PP-elastomer (Versify®2200 present at 49 percent) with anti-block and slip additives, and blends of EMA (Elvaloy®1330AC present at 49 percent) and PP-elastomer (Versify®2200 present at 49 percent) also with anti-block and slip additives. PP-elastomers such as Versify® from Dow, Vistamaxx® from Exxon, and Notio® from Mitsui are also suitable.

In addition to heat sealability, the skin layers 40, 42 preferably also provide sound absorbing properties to the film 28. In such an embodiment, each of the skin layers 40, 42 comprises a vinyl-bond rich triblock copolymer, such as Hybrar®, to enhance mechanical properties and sound absorbing properties of the film. For example, each of the skin layers 40, 42 is formed from a blend of a non-hydrogenated styrene isoprene copolymer (Hybrar® 5127), a PP-elastomer (Vistamaxx®), and an EMA (Lotryl® 20MA08).

In a preferred embodiment, the seven-layer film 28 is a sound absorbing film, in which at least one of the layers comprises a vinyl-bond rich triblock copolymer, such as a non-hydrogenated vinyl-bond rich styrene-isoprene-styrene (SIS) block copolymer, e.g. Hybrar® 5125 and 5127, or hydrogenated vinyl-bond rich styrene-isoprene-styrene (SEPS) block copolymer, e.g. Hybrar® 7125.

Referring back to FIG. 1, each of the walls 16, 18 is heat sealed to the non-woven layers 24, 26. In this embodiment, a surface of the wall 16 facing the non-woven layer 24 is heat sealed to the adjacent surface of the non-woven layer 24 along its entire surface area, thereby forming a thermal laminate. Similarly, the wall 18 and the non-woven layer 26 are heat sealed along their entire surface, also forming a thermal laminate. In other embodiments, the wall and the non-woven layer may be heat sealed along their peripheral edges only, or the pouch 10 may only include one non-woven layer or no non-woven layer. Alternatively, the wall and the non-woven layer can be laminated using an adhesive between them.

Preferably, at least one of the non-woven layers 24, 26 is formed of a sound absorbing non-woven material. The sound absorbing non-woven material is formed from fibers comprising a vinyl-bond rich triblock copolymer, such as a non-hydrogenated vinyl-bond rich styrene-isoprene-styrene (SIS) block copolymer, e.g. Hybrar® 5125 and 5127, or a hydrogenated vinyl-bond rich styrene-isoprene-styrene (SEPS) block copolymer, e.g. Hybrar® 7125.

In one embodiment, the fibers are formed of a blend of Hybrar®, and PE or PP. In another embodiment, the fibers are formed of a blend of a hydrogenated vinyl-bond rich styrene-isoprene-styrene (SEPS) block copolymer, such as Hybrar® 7125, and at least one other polymer. For example, the fibers are formed from a blend of Hybrar® 7125 and a hydrogenated poly(styrene-isoprene-styrene) (SEPS), such as Septon®, which is available from Kuraray Co. Ltd. The Septon® SEPS is distinguishable from Hybrar® 7125 in that the midblock of the Septon® SEPS is a hydrogenated polyisoprene whereas the midblock of Hybrar® 7125 is hydrogenated vinyl-polyisoprene. Surprisingly, test results indicate that the vinyl-bond rich Hybrar® 7125 provides significantly better sound absorbing properties than Septon® SEPS. In another example, the fibers are formed from a blend of a low molecular weight hydrogenated vinyl-bond rich styrene-isoprene-styrene (SEPS) block copolymer, such as HG664 from Kuraray Co. Ltd., a Septon® SEPS block copolymer, and a low molecular weight PP.

In some embodiments, the fibers have a sheath 44/core 46 structure (FIG. 3), in which the core 46 is formed of Hybrar® and the sheath 44 is formed of PE or PP. In such an embodiment, the tacky Hybrar® core is enclosed by the sheath, which is advantageous during manufacturing of non-woven materials from the fibers. The sound absorbing non-woven material can be formed by carding and dry-laying such fibers. Alternatively, the non-woven material can be formed via melt blown or spunbond technologies.

Although the embodiment shown in FIG. 1 is a one-piece ostomy appliance with a closed-end pouch, the above discussed sound absorbing multilayer film and sound absorbing non-woven material can be used to make other types of ostomy appliances, such as two-piece ostomy appliances and drainable ostomy pouches.

Examples and Test Results

Monolayer Films

Four different sample monolayer films comprising a triblock copolymer were prepared, each of which having a thickness of about 4 mil. Tangent delta values of each film, which correlate to sound absorbing capabilities, were measured, and compared against a control film. The control film was a six-layer film having a thickness of about 2.24 mil (57 μm) and including a seal layer/tie layer/barrier layer/tie layer/inner layer/skin layer construction. The seal layer has a thickness of about 22.5 μm and comprises a blend of 97.5% wt. EVA copolymer (Escorene® FL00218 available from ExxonMobil Corporation) and 2.5% wt. anti-block/slip additive (Polybatch® SAB1982VA available from Schulman Inc.) Each of the tie layers has a thickness of about 4 μm and comprises a blend of 80% wt. EMA (Lotryl® 18MA02) and 20% wt. MAH grafted LLDPE (Bynel® CXA41E710.) The barrier layer has a thickness of about 4 μm and comprises a blend of 85% wt. of an amorphous polyamide (Selar® PA3426R) and 15% wt. functionalized rubber blend (Lotader® 4720.) The inner layer has a thickness of about 18 μm and comprises a blend of 87% wt.-89.5% wt. EVA copolymer (Escorene® FL00218) and 10.5% wt.-13% wt. Schulman® T92030 Beige.

Further, each of the sample films was heat sealed with a PE non-woven and made into a pouch to measure flatulence noise insulation properties. The PE non-woven was heat sealed to the sample film along its entire surface area, thereby forming a thermal laminate, whereas the control film was laminated to the PE non-woven layer by an adhesive applied along the entire surface area between the PE non-woven layer and the control film, thereby forming an adhesive laminate. The adhesive was applied at a thickness of about 1.1 mil. The flatulence noise emission levels were measured with gas only, and gas and water in the pouch.

TABLE 1
Tangent Delta Values of Monolayer Films comprising Hybrar ®

| | | | | | | | | | | | Flatulence | |
|---|---|---|---|---|---|---|---|---|---|---|---|---|
| | | | | | | | | | | | Noise | |
| | | | | | | | | | | | SPL(A)dB(A)* | |
| Film | | Tangent Delta (at T ° C.) | | | | | | | | | Gas | Gas + |
| Code | Composition | −30 | −20 | −10 | 0 | 10 | 20 | 23 | 30 | 40 | Only | Water |
| Control | 5-layer film | 0.21 | 0.22 | 0.18 | 0.14 | 0.11 | 0.07 | 0.07 | 0.06 | 0.05 | 65.0 | 62.0 |
| Sample | Hybrar ® 7125 | 0.09 | 0.21 | 0.54 | 0.63 | 0.40 | 0.23 | 0.20 | 0.15 | 0.11 | 59.6 | 58.7 |
| 280-3 | (49% wt.), | | | | | | | | | | | |
| | Vistamaxx ® | | | | | | | | | | | |
| | 3020(49% wt), | | | | | | | | | | | |
| | Polybatch ® | | | | | | | | | | | |
| | SAB1982 | | | | | | | | | | | |
| | (2% wt) | | | | | | | | | | | |
| Sample | Hybrar ® 7125 | 0.08 | 0.20 | 0.50 | 0.59 | 0.36 | 0.22 | 0.19 | 0.14 | 0.10 | 59.8 | 59.3 |
| 280-4A | (48.5% wt.), | | | | | | | | | | | |
| | Vistamaxx ® | | | | | | | | | | | |
| | 3020 | | | | | | | | | | | |
| | (48.5% wt.), | | | | | | | | | | | |
| | Polybatch ® | | | | | | | | | | | |
| | SAB1982 | | | | | | | | | | | |
| | (2% wt.), | | | | | | | | | | | |
| | FP40 (1% wt.) | | | | | | | | | | | |
| Sample | Hybrar ® 5127 | 0.08 | 0.08 | 0.07 | 0.10 | 0.35 | 0.64 | 0.56 | 0.36 | 0.20 | 58.4 | 55.3 |
| 280-5 | (49% wt.), | | | | | | | | | | | |
| | Lotryl ® | | | | | | | | | | | |
| | 20MA08 | | | | | | | | | | | |
| | (49% wt.), | | | | | | | | | | | |
| | Polybatch ® | | | | | | | | | | | |
| | SAB1982 | | | | | | | | | | | |
| | (2% wt.) | | | | | | | | | | | |
| Sample | Hybrar ® 5127 | 0.06 | 0.07 | 0.11 | 0.13 | 0.28 | 0.47 | 0.42 | 0.31 | 0.18 | 59.8 | 57.5 |
| 280-8 | (49% wt.), | | | | | | | | | | | |
| | Vistamaxx ® | | | | | | | | | | | |
| | 3980(49% wt), | | | | | | | | | | | |
| | Polybatch ® | | | | | | | | | | | |
| | SAB1982 | | | | | | | | | | | |
| | (2% wt.) | | | | | | | | | | | |

*SPL(A) dB(A): noise spectrum between 8 and 16000 Hz.

As can be seen from the results in Table 1, the pouches made using the films comprising a vinyl-bond rich triblock copolymer (Hybrar® 7125 and 5127) emit significantly less flatulence noise, and thus have improved sound insulation properties when compared to the pouch made using the control film. Further, the films comprising the Hybrar® have substantially higher tangent delta values around operating temperatures of ostomy pouches (e.g. 10° C., 20° C., 23° C., and 30° C.), which correlate to better sound absorbing properties.

Non-Wovens

Five different non-woven samples were prepared and tested for sound insulation characteristics. Non-woven Sample#1 included a layer of non-woven having a basis weight of about 20 gsm of fibers formed from about 100% Septon® SEPS block copolymer, and a layer of non-woven having a basis weight of about 20 gsm of bi-component fibers including a PP core and high density polyethylene (HDPE) sheath. Non-woven Sample#1 is prepared by using a preformed PP/HDPE bi-component fiber non-woven and laying Septon® SEPS block copolymer fibers on the PP/HDPE non-woven layer to form the Septon® SEPS block copolymer fiber non-woven layer having a basis weight of about 20 gsm.

Non-woven Sample#2 included the same preformed PP/HDPE non-woven used for Non-woven Sample#1, and a layer of non-woven having a basis weight of about 20 gsm of fibers formed from a polymer blend including about 90 wt. % Septon® SEPS block copolymer and about 10 wt. % PP.

Non-woven Sample#3 was made using fibers formed from a polymer blend including about 70 wt. % Septon® SEPS block copolymer and 30 wt. % PP. Non-woven Sample#3 had a basis weight of about 40 gsm of the polymer blend fibers.

Non-woven Sample#4 was made using fibers formed from a polymer blend including about 47.5 wt. % HG664 (low molecular weight hydrogenated vinyl-bond rich styrene-isoprene-styrene (SEPS) block copolymer), about 47.5 wt. % Septon® SEPS block copolymer, and about 5 wt. % low molecular weight PP. Non-woven Sample#4 had a basis weight of about 40 gsm of the polymer blend fibers.

Non-woven Sample#5 included the same preformed PP/HDPE bi-component fiber non-woven used for Non-woven Sample#1, and a layer of non-woven made using 20 gsm of fibers formed from a polymer blend including about 30 wt. % Hybrar® 7125 (hydrogenated vinyl-bond rich styrene-isoprene-styrene (SEPS) block copolymer) and about 70 wt. % Septon® SEPS block copolymer.

The non-woven samples were laminated with a six-layer film to prepare eight different laminate samples. The six-layer film has a thickness of about 2.24 mil (57 μm) and a seal layer/tie layer/barrier layer/tie layer/inner layer/skin layer construction. The seal layer has a thickness of about 22.5 μm and comprises a blend of 97.5% wt. EVA copolymer (Escorene® FL00218 available from ExxonMobil Corporation) and 2.5% wt. anti-block/slip additive (Polybatch® SAB1982VA available from Schulman Inc.) Each of the tie layers has a thickness of about 4 μm and comprises a blend of 80% wt. EMA (Lotryl® 18MA02) and 20% wt. MAH grafted LLDPE (Bynel® CXA41E710.) The barrier layer has a thickness of about 4 μm and comprises a blend of 85% wt. of an amorphous polyamide (Selar® PA3426R) and 15% wt. functionalized rubber blend (Lotader® 4720.) The inner layer has a thickness of about 18 μm and comprises a blend of 87% wt.-89.5% wt. EVA copolymer (Escorene®FL00218) and 10.5% wt.-13% wt. Schulman® T92030 Beige.

A control laminate sample (295-1) was prepared by laminating a non-woven having a basis weight of about 40 gsm of PE fibers and the six-layer film using an adhesive having a thickness of about 1.1 mil between the two layers. This adhesive was also used for other laminate samples (Laminate Samples 295-2, 295-2Rev, 295-3, 295-3Rev, 295-4, 295-5, 295-6, 295-6Rev.)

Laminate Sample 295-2 was prepared by laminating Non-woven Sample#1 and the six-layer film with the adhesive between the layers. The six-layer film was laminated on the Septon® SEPS block copolymer fiber side of the Non-woven Sample#1. Laminate Sample 295-2Rev was also prepared by laminating Non-woven Sample#1 and the six-layer film with the adhesive between the layers. However, for this sample, the six-layer film was laminated on the PP/HDPE bi-component fiber side of Non-woven Sample#1.

Laminate Sample 295-3 was prepared by laminating Non-woven Sample#2 and the six-layer film with the adhesive between the layers. The six-layer film was laminated on the Septon® SEPS block copolymer/PP blend fiber side of Non-woven Sample#2. Laminate Sample 295-3Rev was also prepared by laminating Non-woven Sample#2 and the six-layer film with the adhesive between the layers. For this sample, the six-layer film was laminated on the PP/HDPE bi-component fiber side of Non-woven Sample#2.

Laminate Sample 295-4 was prepared by laminating Non-woven Sample#3 and the six-layer film with the adhesive between the layers.

Laminate Sample 295-5 was prepared by laminating Non-woven Sample#4 and the six-layer film with the adhesive between the layers.

Laminate Sample 295-6 was prepared by laminating Non-woven Sample#5 and the six-layer film with the adhesive between the layers. The six-layer film was laminated on the Hybrar® 7125/Septon® SEPS block copolymer blend fiber side of Non-woven Sample#5. Laminate Sample 295-6Rev was also prepared by laminating Non-woven Sample#5 and the six-layer film with the adhesive between the layers. For this sample, the six-layer film was laminated on the PP/HDPE bi-component fiber side of Non-woven Sample#5.

The laminate samples were tested according to ASTM E2611-09 (Standard Test Method for Measurement of Normal Incidence Sound Transmission of Acoustical Materials Based on the Transfer Matrix Method). In this test, a sound source (e.g. loudspeaker) is mounted at one end of an impedance tube, and a laminate sample is placed in a holder in the tube at a distance away from the sound source. The loudspeaker generates broadband, stationary random sound waves that propagate as plane waves. The plane waves hit the laminate sample with part of the waves reflected back into the source tube, part absorbed by the laminate sample, and part passing through the laminate material to a receiving tube. By measuring the sound pressure at four fixed locations (two in the source tube and two in the receiving tube) and calculating the complex transfer function using a four-channel digital frequency analyzer, the transmission loss of the laminate sample is determined.

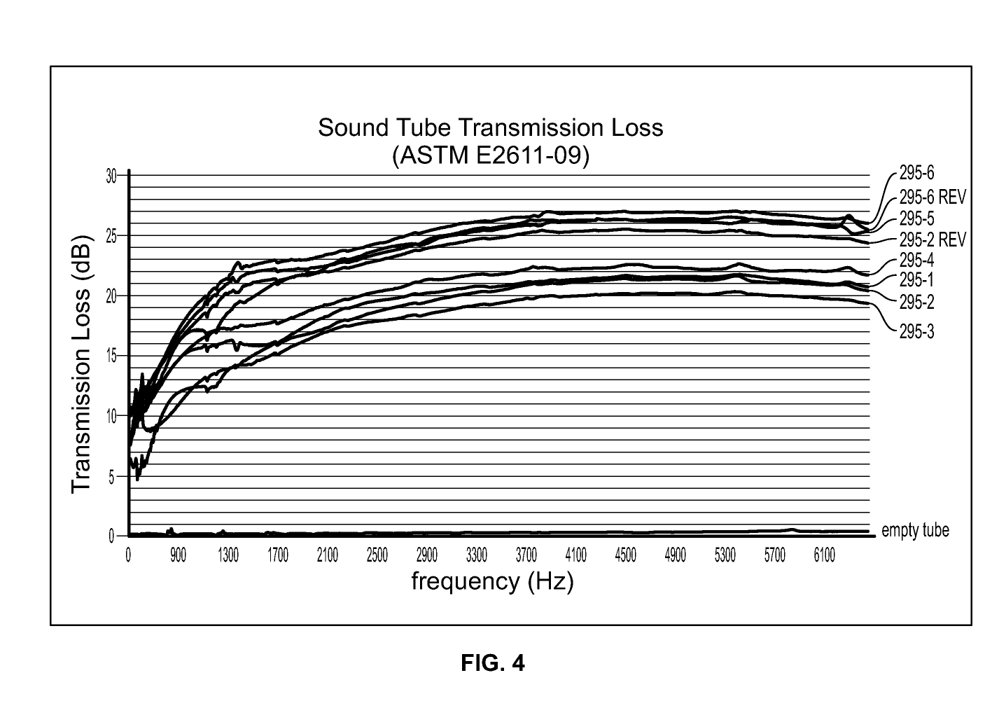

The sound tube transmission loss test data for laminate samples are plotted and shown in FIG. 4. Transmission loss expressed in decibel (dB) shows the degree of sound reduced or absorbed by the laminate samples. The most audible range of human hearing is between about 1,000 Hz to 4,000 Hz. As shown in FIG. 4, Laminate Samples 295-5, 295-6 and 295-6Rev provide the best sound reduction/absorption characteristics with about 5-6 dB sound reduction above 1300 Hz frequency. For the ostomy pouch application, the sound transmission loss of about 5-6 dB can provide a significant reduction of embarrassing flatus gas noise.

Ostomy Pouch Embodiments

In embodiment 1, an ostomy pouch of FIG. 1 was made using a multilayer film including at least one layer comprising a non-hydrogenated vinyl-bond rich triblock copolymer, such as Hybrar® 5125 or Hybrar® 5127, and a non-woven material formed from fibers comprising the non-hydrogenated vinyl-bond rich triblock copolymer. Each of the walls 16, 18 of the pouch 12 is formed from a seven-layer film having the construction shown in FIG. 2. In this embodiment, the seven-layer film has a thickness of about 4 mil, which includes an odor barrier layer 30 having a thickness of about 4 μm, two tie layers 32, 34, each of which having a thickness of about 3 μm, two inner layers 36, 38, each of which having a thickness of about 10 μm, and two skin layers, each of which having a thickness of about 40 μm.

The odor barrier layer 30 is formed from a blend including about 85% wt. amorphous nylon (Selar® PA3426R) and about 15% wt. functionalized rubber blend or compound (Lotader® 4720). Each of the tie layers 32, 34 is formed of a maleated EMA (Lotader® 4503). Each of the inner layers 36, 38 is formed from a blend of a vinyl-bond rich triblock copolymer (Hybrar®) and a PP-elastomer) (Vistamaxx®. Preferably the vinyl-bond rich triblock copolymer is a non-hydrogenated SIS block copolymer (Hybrar® 5125 or Hybrar® 5127.) Each of the skin layers 40, 42 is formed from a blend of a non-hydrogenated vinyl-bond rich SIS block copolymer (Hybrar® 5125 or Hybrar® 5127), a PP-elastomer (Vistamaxx®), and an EMA copolymer (Lotryl® 20MA08.) Preferably, the polymer blends for the inner layers and the skin layers comprise more than about 50% wt. of the non-hydrogenated vinyl-bond rich SIS block copolymer to maximize sound absorbing properties of the film. In one embodiment, at least one of the inner layers and skin layers comprise more than about 60% wt. of the non-hydrogenated vinyl-bond rich SIS block copolymer.

In this embodiment, the sound absorbing non-woven layers 24, 26 are provided on each pouch wall to further improve sound insulation characteristics of the ostomy pouch. The non-woven layers, 24, 26 are directly attached to the first and second walls 16, 18, respectively, via heat sealing. Alternatively, the non-woven layers can be attached to the first and second walls using an adhesive.

The sound absorbing non-woven layers 24, 26 are formed from fibers comprising a non-hydrogenated vinyl-bond rich triblock copolymer (Hybrar® 5125 or Hybrar® 5127.) In this embodiment, each of the non-woven layers 24, 26 is a sound absorbing non-woven material comprising about 40 gsm of fibers including a core formed of Hybrar® 5127 and a sheath formed of PE or PP.

Thus, the pouch includes a total of four sound absorbing layers, to wit, two walls formed from the seven-layer sound absorbing film and two non-woven layers including sound absorbing fibers. As such, the pouch provides substantially improved flatulence insulating properties compared to conventional ostomy pouches.

In embodiment 2, an ostomy pouch was constructed similarly as the ostomy pouch of embodiment 1, except the sound absorbing non-woven layers 24, 26 are made using fibers formed from a polymer blend comprising about 30 wt. % to about 70 wt. % of low molecular weight hydrogenated vinyl-bond rich styrene-isoprene-styrene block copolymer and a SEPS block copolymer (e.g. Septon® SEPS block copolymer.)

In embodiment 3, an ostomy pouch was constructed similarly as the ostomy pouch of embodiment 1, except the sound absorbing non-woven layers 24, 26 are made using fibers formed from a polymer blend comprising about 20 wt. % to about 70 wt. % of hydrogenated vinyl-bond rich styrene-isoprene-styrene block copolymer (e.g. Hybrar® 7125.)

In embodiment 4, an ostomy pouch was constructed similarly as the ostomy pouch of embodiment 1, except at least one of the inner and skin layers 36, 38, 40, 42 of the multilayer film comprises hydrogenated vinyl-bond rich styrene-iroprene-styrene block copolymer (e.g. Hybrar® 7125), and the sound absorbing non-woven layers 24, 26 are made using fibers formed from a polymer blend comprising about 20 wt. % to about 70 wt. % of hydrogenated vinyl-bond rich styrene-isoprene-styrene block copolymer (e.g. Hybrar® 7125.)

In the present disclosure, the words “a” or “an” are to be taken to include both the singular and the plural. Conversely, any reference to plural items shall, where appropriate, include the singular. All of the concentrations noted herein as percentage are percent by weight unless otherwise noted.

From the foregoing it will be observed that numerous modifications and variations can be effectuated without departing from the true spirit and scope of the novel concepts of the present invention. It is to be understood that no limitation with respect to the specific embodiments illustrated is intended or should be inferred. The disclosure is intended to cover by the appended claims all such modifications as fall within the scope of the claims.

1. A sound insulating ostomy pouch, comprising:
a first wall,
a second wall, wherein the first wall and the second wall are sealed along their peripheral edges to define a cavity; and
at least one sound absorbing non-woven layer including fibers formed from a polymer blend comprising a vinyl-bond rich triblock copolymer, wherein the at least one sound absorbing non-woven layer is attached to the first wall.

2. The sound insulating ostomy pouch of claim 1, wherein at least one of the first wall and the second wall is formed from a sound absorbing film including a vinyl-bond rich triblock copolymer.

3. The sound insulating ostomy pouch of claim 1, wherein the sound absorbing film is a multilayer film including at least one layer containing more than 50% wt. of a non-hydrogenated vinyl-bond rich SIS block copolymer.

4. The sound insulating ostomy pouch of claim 1, wherein the sound absorbing film is a seven-layer film including an odor barrier layer, two tie layers, two inner layers, and two skin layers, wherein at least one of the skin layers contains more than 50% wt. of a non-hydrogenated vinyl-bond rich SIS block copolymer.

5. The sound insulating ostomy pouch of claim 4, wherein at least one of the inner layers contain a vinyl-bond rich triblock copolymer.

6. The sound insulating ostomy pouch of claim 5, wherein at least one of the inner layers contains more than 50% wt. of a non-hydrogenated vinyl-bond rich SIS block copolymer.

7. The sound insulating ostomy pouch of claim 1, wherein the fibers are formed from a polymer blend comprising a non-hydrogenated vinyl-bond rich SIS block copolymer and a polyethylene or a polypropylene.

8. The sound insulating ostomy pouch of claim 1, wherein the fibers are formed from a polymer blend comprising about 20 wt. % to about 70 wt. % of a hydrogenated vinyl-bond rich styrene-isoprene-styrene block copolymer.

9. The sound insulating ostomy pouch of claim 1, wherein the pouch includes a first sound absorbing non-woven layer and a second sound absorbing non-woven layer, wherein the first sound absorbing non-woven layer is heat sealed to the first wall and the second sound absorbing non-woven layer is heat sealed to the second wall, wherein each of the first sound absorbing non-woven layer and the second sound absorbing non-woven layer includes fibers formed from a polymer blend comprising a hydrogenated vinyl-bond rich styrene-isoprene-styrene block copolymer.

10. The sound insulating ostomy pouch of claim 1, wherein the at least one sound absorbing non-woven layer is heat sealed to the first wall.

11. The sound insulating ostomy pouch of claim 1, wherein the at least one sound absorbing non-woven layer is attached to the first wall via an adhesive.

12. A sound absorbing non-woven material for ostomy appliances, comprising fibers including a non-hydrogenated vinyl-bond rich SIS block copolymer.

13. The sound absorbing non-woven material of claim 12, wherein the sound absorbing non-woven material comprises fibers formed from a blend of the non-hydrogenated vinyl-bond rich SIS block copolymer, and a polyethylene or a polypropylene.

14. The sound absorbing non-woven material of claim 12, wherein the sound absorbing, non-woven material comprises fibers having a sheath/core construction, wherein the sheath is formed of a polyethylene or a polypropylene, and the core is formed of the non-hydrogenated vinyl-bond rich SIS block copolymer.

15. The sound absorbing non-woven material of claim 12, wherein the non-woven material has a basis weight between about 20 gsm and about 60 gsm of fibers.

16. A sound absorbing non-woven material for ostomy appliances, comprising fibers are formed from a polymer blend comprising a hydrogenated vinyl-bond rich styrene-isoprene-styrene block copolymer.

17. The sound absorbing non-woven material of claim 16, wherein the fibers are formed from a polymer blend comprising about 20 wt. % to about 70 wt. % of the hydrogenated vinyl-bond rich styrene-isoprene-styrene block copolymer.

18. The sound absorbing non-woven material of claim 16, wherein the non-woven material has a basis weight between about 20 gsm and about 60 gsm of fibers.

19. A sound absorbing film for ostomy appliances, comprising at least one layer comprising at least 50% wt. of a non-hydrogenated vinyl-bond rich SIS block copolymer.

20. The sound absorbing film of claim 19, wherein the sound absorbing film is a multilayer film including an odor barrier layer and a first skin layer and a second skin layer, wherein at least one of the first skin layer and the second skin layer comprises at least 50% wt. of the non-hydrogenated vinyl-bond rich SIS block copolymer.

21. The sound absorbing film of claim 20, wherein the multilayer film further includes a first inner layer and a second inner layer, wherein at least one of the first inner layer and the second inner layer includes a vinyl-bond rich triblock copolymer.

22. The sound absorbing film of claim 21, wherein at least one of the first inner layer and the second inner layer comprises at least 50% wt. of the non-hydrogenated vinyl-bond rich SIS block copolymer.

23. The sound absorbing film of claim 19, wherein the sound absorbing film is a seven-layer film including an odor barrier layer, two tie layers, two inner layers, and two skin layers, wherein at least one of the skin layers contains at least 50% wt. of the non-hydrogenated vinyl-bond rich SIS block copolymer, and at least one of the inner layers contains at least 50% wt. of the non-hydrogenated vinyl-bond rich SIS block copolymer.

24. A sound absorbing multilayer film for ostomy appliances, comprising at least one layer comprising a hydrogenated vinyl-bond rich SIS block copolymer.

25. The sound absorbing multilayer film of claim 24, wherein the sound absorbing film is a multilayer film including an odor barrier layer and a first skin layer and a second skin layer, wherein at least one of the first skin layer and the second skin layer comprises the hydrogenated vinyl-bond rich SIS block copolymer.

26. The sound absorbing multilayer film of claim 25, wherein the multilayer film further includes a first inner layer and a second inner layer, wherein at least one of the first inner layer and the second inner layer includes a vinyl-bond rich triblock copolymer.

27. The sound absorbing multilayer film of claim 26, wherein at least one of the first inner layer and the second inner layer comprises the hydrogenated vinyl-bond rich SIS block copolymer.

28. The sound absorbing multilayer film of claim 24, wherein the sound absorbing film is a seven-layer film including an odor barrier layer, two tie layers, two inner layers, and two skin layers, wherein at least one of the skin layers contains the hydrogenated vinyl-bond rich SIS block copolymer, and at least one of the inner layers contains the hydrogenated vinyl-bond rich SIS block copolymer.
